(19) United States
(12) Patent Application Publication
(10) Pub. No.: US 2022/0127674 A1
(43) Pub. Date: Apr. 28, 2022

(54) METHODS FOR DETECTING CIRCULATING STROMAL CELLS

(71) Applicant: Stichting Katholieke Universiteit, Nijmegen (NL)

(72) Inventors: Wilhelmus Theodorus Stefanus HUCK, Nijmegen (NL); Kinga MATULA, Nijmegen (NL); Francesca Maria RIVELLO, Nijmegen (NL)

(21) Appl. No.: 17/082,489

(22) Filed: Oct. 28, 2020

(51) Int. Cl. C12Q 1/6886; C12Q 1/6818; B01L 3/00; G01N 33/533

(52) U.S. Cl. CPC C12Q 1/6886; C12Q 1/6818; B01L 2300/0829; G01N 33/533; B01L 3/502792

(57) ABSTRACT

Novel methods for detecting circulating stromal cells are provided. The methods comprise the steps of incubating a sample, and comparing the pH and/or concentration of at least one molecule selected from the group consisting of lactic acid, lactate ions, and pyruvate ions, determined for the incubated volume to the pH and/or concentration of said at least one molecule, within said sample, wherein a decrease in pH and/or an increase in concentration of said at least one molecule, indicates said at least one circulating stromal cell is present in said sample.

### TECHNICAL FIELD

This disclosure relates to methods for detecting circulating stromal cells.

### BACKGROUND

In addition to malignant cells, all solid tumors contain a variety of nonmalignant cancer-associated stromal cells, which include, but are not limited to, endothelial cells (ECs), mesenchymal stem cells (MSCs), cancer-associated fibroblasts (CAFs), pericytes, and immune cells. These non-malignant cancer-associated stromal cells can make up more than 50% of the tumor mass. These stromal cells are increasingly considered essential for nutrient and growth factor delivery, inducement of the epithelial-to-mesenchymal transition, and tumor growth. Furthermore, their propensity to dissociate from the primary tumor (at a rate of about 1 million cells per day) gives them an important role in metastatic dissemination.

Importantly, cancer-associated metabolic remodeling occurs in cancer-associated cells such as stromal cells that are within a tumor microenvironment. In particular, stromal metabolic remodeling was reported in e.g. CAFs as a result of hypoxic or oxidative stress induced by cancer cells. Cross-talk between e.g. CAFs and cancer cells at the tumor microenvironment triggers anaerobic glycolysis, leading to the production of, inter alia, lactate and pyruvate.

When stromal cells are not part of a tissue, for example after dissociating from the tumor, said stromal cells are known as “circulating stromal cells”. The prior art does not discuss whether or not after dissociation with the tumor microenvironment that stimulates cancer-associated metabolic remodeling, said remodeling still occurs in circulating stromal cells. Hence, the prior art is entirely silent on whether circulating stromal cells produce acids such as lactate and pyruvate.

Circulating stromal cells are present in very low numbers per unit volume of body fluid, e.g. blood, in a subject, making their detection and analysis challenging, while said detection and analysis are highly relevant for diagnostic and prognostic purposes. However, relatively few studies have reported the isolation and characterization of circulating stromal cells. One of the difficulties is that circulating stromal cells exhibit a very high phenotypic diversity and do not have general markers. As such, there is an urgent need for a more general method for the detection of circulating stromal cells. Furthermore, it is desired that such a method be easy to implement, cheap, achieve a high throughput, and/or use few chemicals.

### SUMMARY

The disclosure provides a method for detecting at least one circulating stromal cell in a sample of about 10 pL to about 10 nL of a fluid, wherein said sample comprises at least one cell, and wherein the pH and/or concentration of at least one molecule selected from the group consisting of lactic acid, lactate ions, and pyruvate ions, within said sample, has been determined before step (c) of said method; wherein said method comprises the steps of:

(a) incubating said sample at a temperature from about 4° C. to about 42° C. for at least 1 minute to provide an incubated volume;
(b) determining the pH and/or concentration of said at least one molecule, within said incubated volume; and
(c) comparing the pH and/or concentration of said at least one molecule determined in step (b) to the pH and/or concentration of said at least one molecule, within said sample;
wherein a decrease in pH and/or an increase in concentration of said at least one molecule, indicates said at least one circulating stromal cell is present in said sample.

### BRIEF DESCRIPTION OF THE FIGURES

FIG. 1 depicts an experimental workflow, according to an embodiment of the disclosure, for detecting circulating stromal cells from patient liquid biopsies using a droplet- based microfluidic device.

FIG. 1A depicts lysis of red blood cells.

FIG. 1B depicts negative depletion of the sample from white blood cells highly expressing CD45 antigen at the surface.

FIG. 1C depicts optional staining with fluorescently labeled anti-EpCAM and anti-CD45 antibodies.

FIG. 1D depicts emulsification of sample comprising at least one cell in water-in-oil droplets, with a pH ratiometric dye.

FIG. 1E depicts incubation of the emulsion at 37° C., which allows cells to consume available glucose and acidify their extracellular microenvironment.

FIG. 1F shows the reinjection of the droplets into the co-flow device and online measurement of the ratio of the fluorescence intensity at 580/630 nm, followed by sorting induced by electrocoalescence. For stained samples, EpCAM and CD45 markers are measured simultaneously with pH.

FIG. 2 depicts the enumeration of highly metabolically active cells (hm-cells) for healthy donors and metastatic prostate cancer patients (normalized to 7.5 ml of blood).

FIG. 2A shows the comparison of the number of cells acidifying below pH <6.5 for 26 healthy donors (HD) and 56 metastatic prostate cancer patients. The median number of events is shown as a black line: 3 hm-cells for healthy donors and 11 hm-cells for patients.

FIG. 2B depicts the number of positive events detected for prostate cancer patients determined for droplets containing cells with pH <6.5, 6.8, and 7.0. Median values are 11 hm-cells for pH <6.5, 90 cells for pH <6.8, and 100 cells for pH <7.0. NS means “not significant”.

FIG. 2C depicts the number of EpCAM+ cells detected in patient samples established for droplets acidified to pH <6.5, 6.8, and 7.0. Comparison of median between groups (A to C) was performed by Mann-Whitney test (**P <0.01 and***P<0.001).

FIGS. 2D-F depict the total number of EpCAM+ and EpCAM− cells for individual prostate cancer patients' samples encapsulated in droplets acidified to pH <6.5, 6.8, and 7.0, respectively. Plots presented in FIGS. 2A and 2B were prepared for 56 patients and 26 healthy donors, while plots presented in FIGS. 2C-F were determined for 26 stained patient samples.

FIG. 3 depicts the correlation between hm-cells and survival probability.

FIG. 3A shows a Kaplan-Meier plot for 54 metastatic prostate cancer patients stratified using a cutoff value of five or less hm-cells (the evolution of the number of patients is provided in the table below the figure). Censored patients are marked with “+” over the curves. Twenty-six patients had zero to five hm-cells, and 28 patients had more than five hm-cells. P=0.0217 was obtained by the log-rank (Mantel-Cox) test. Median survival was 229 days for the patients with more than five hm-cells and was not reached for the patients with zero to five hm-cells.

FIG. 3B shows the size evaluation of detected hm-cells for patients with ≤5 (1556 cells) or >5 hm-cells (418 cells) shown as median with interquartile ranges.

FIG. 4 depicts the analysis of the single-cell mRNA expression of hm-cells compared to patient-derived white blood cells.

FIG. 4A shows a t-distributed stochastic neighbor embedding (t-SNE plot) visualizing the two clusters in which all the cells from the six 96-well plates were divided into. Each dot corresponds to a single cell. n=152 cells.

FIG. 4B shows a t-SNE plot showing origin (i.e., plate number) for all the cells: one patient-derived white blood cell plate (n=59 cells) and five patient-derived hm-cell plates (Pt1, Pt2, Pt3, Pt4, and Pt5) (n=93 cells). Each dot corresponds to one single cell.

FIG. 4C shows a heatmap representing the expression of the 131 differentially expressed genes (DEGs) characterizing the two clusters, of which 69 are up-regulated in cluster 0 and 62 are up-regulated in cluster 1. Each line corresponds to a single cell, and each column corresponds to one of the DEGs. The legend on the left of the heatmap shows the cluster and the plate to which each cell belongs to.

FIG. 4D shows the pathway analysis classification of the DEGs significantly up-regulated in cluster 1 (containing 80% of the hm-cells).

FIG. 4E shows the classification of pathways according to the KEGG database of the DEGs significantly up-regulated in cluster 0 (white blood cells cluster, gray) and in cluster 1 (hm-cells cluster, violet).

FIG. 5 depicts t-SNE plots with the cumulative expression of known genes for hm-cells compared to WBCs.

FIG. 5A shows prostate cancer-related genes: AR, ERG, HOXB2, KLK3, PCA3, and TMPRSS2.

FIG. 5B shows mesenchymal stem cell-related genes: ANPEP, CD24, CD9, ITGB1,and TFRC.

FIG. 5C shows endothelial-related genes: CD34, IGFBP7, MCAM, and VWF.

FIG. 5D depicts epithelial cell adhesion molecule (EPCAM).

FIG. 5E depicts fibroblast-related genes: EGF, FGF2, FGFR1, FN1, PDGFB, PDGFC, and TGFB1.

FIG. 5F shows vascular cancer-associated fibroblast-related genes: ESAM, GNG11, HIGD1B, COX412, CYGB, GJA4, and ENG.

### DETAILED DESCRIPTION

The method of the disclosure is based on the judicious insight that circulating stromal cells continue to produce acids such as lactate and pyruvate, even while the circulating stromal cells are not associated with the tumor microenvironment. This is surprising, since the prior art only discloses that stromal cells in direct contact with the tumor microenvironment can produce significant amounts of acids such as lactate and pyruvate. Since circulating stromal cells are not in direct contact with the tumor microenvironment, it was entirely unknown whether these circulating stromal cells produce significant amounts of acids such as lactate and pyruvate.

This insight allows circulating stromal cells to be detected with a method according to the disclosure, which is based on detecting a decrease in pH and/or an increase in the concentration of at least one molecule selected from the group consisting of lactic acid, lactate ions, and pyruvate ions, in a sample having a small volume, after a brief incubation step.

The methods of the disclosure achieve one or more of the abovementioned needs and desires. In particular, said methods are general, because circulating stromal cells in general were shown to produce acids such as lactate and/or pyruvate. In addition, the methods of the disclosure are easy to implement, cheap, achieve a high throughput, and/or use few chemicals.

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the methods of the disclosure will be apparent from the following detailed description and figures, and from the claims.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

A “circulating stromal cell” as used herein refers to stromal cells that are derived from a tumor tissue, but are not part of a tumor tissue. For example, a circulating stromal cell may be a stromal cell that has dissociated from a tumor environment, and has for example entered the blood stream. Similarly, “circulating stromal cell” may herein also refer to stromal cells that have dissociated from a tumor cultured in vitro, for example an organoid. As such, it will be understood that a circulating stromal cell as used herein is not necessarily in circulation, but may also for example be isolated in a small volume. “Circulating” in this respect is understood to relate to the fact that the circulating stromal cell is not part of a tissue. Circulating stromal cells include, but are not limited to, circulating endothelial cells, circulating mesenchymal stem cells, circulating cancer-associated fibroblasts, circulating pericytes, and circulating immune cells. Circulating immune cells include circulating cancer-associated macrophage-like cells. In some embodiments, the at least one circulating stromal cell is selected from the group consisting of circulating endothelial cells, circulating mesenchymal stem cells, and circulating cancer-associated fibroblasts. In some embodiments, the circulating stromal cells are highly metabolically active circulating stromal cells. In this sense, “highly metabolically active” indicates that the circulating stromal cells primarily function via an aberrant metabolism compared to healthy cells, showing signs of e.g. anaerobic glycolysis, and produce significantly higher levels of lactate, lactic acid, and/or pyruvate than stromal cells primarily functioning on aerobic glycolysis.

A “subject,” as used herein, includes both humans and other animals, particularly mammals. In some embodiments, the subject is a mammal, for example, a primate. In some embodiments, the subject is a human.

A “W/O emulsion” as used herein, refers to a water-in-oil emulsion. Such emulsions are well-known in the art.

An “organoid” is a miniaturized and simplified version of an organ produced in vitro in three dimensions that shows realistic micro-anatomy. Organoids can be derived from one or a few cells from a tissue, embryonic stem cells or induced pluripotent stem cells, which can self- organize in three-dimensional culture owing to their self-renewal and differentiation capacities. Organoids are typically used to study disease and treatments.

### EMBODIMENTS

The sample is about 10 pL to about 10 nL of a fluid. Such a small volume is advantageous, and allows for readily measuring the pH and/or the concentration of the at least one molecule. In larger volumes, the produced acid would quickly diffuse through the entire volume, hence preventing the build-up of measurable concentrations or a measurable decrease in pH.

In one embodiment, the sample is about 10 pL to about 5 nL of a fluid. In other embodiments, the sample is about 11 pL to about 4 nL of a fluid, about 12 pL to about 3 nL of a fluid, about 13 pL to about 2 nL of a fluid, about 14 pL to about 1 nL of a fluid, about 15 pL to about 900 pL of a fluid, about 16 pL to about 800 pL of a fluid, about 17 pL to about 700 nL of a fluid, about 18 pL to about 600 pL of a fluid, about 19 pL to about 500 pL of a fluid, about 20 pL to about 400 pL of a fluid, about 21 pL to about 300 pL of a fluid, about 22 pL to about 200 pL of a fluid, about 23 pL to about 100 pL of a fluid, about 24 pL to about 90 pL of a fluid, about 25 pL to about 80 pL of a fluid, about 26 pL to about 70 pL of a fluid, about 27 pL to about 60 pL of a fluid, about 28 pL to about 50 pL of a fluid, or about 29 pL to about 40 pL of a fluid. In one embodiment, the sample is about 30 pL of a fluid.

The sample comprises at least one cell. It will be understood that the nature of this at least one cell is typically not known before carrying out step (c) of the method of the disclosure.

It will be understood that the number of cells is not relevant for carrying out the method of the disclosure. If in step (c) of said method no decrease in pH or decrease in the at least one molecule is established, this indicates that no circulating stromal cell is present. If such a decrease or increase is established, then this indicates that at least one circulating stromal cell is present, regardless of the presence of other cells that do not result in such a decrease or increase. Thus, the maximum number of cells in the sample may be merely limited by the volume of the sample and the size of the cells, which can easily be determined by the person skilled in the art.

In some embodiments, the sample comprises at least about 2 cells, at least about 3 cells, at least about 4 cells, at least about 5 cells, at least about 6 cells, at least about 7 cells, at least about 8 cells, at least about 9 cells, at least about 10 cells, at least about 20 cells, at least about 30 cells, at least about 40 cells, at least about 50 cells, at least about 60 cells, at least about 70 cells, at least about 80 cells, at least about 90 cells, or at least about 100 cells.

In some embodiments, the sample comprises at most about 100 cells. In other embodiments, the sample comprises at most about 90 cells, at most about 80 cells, at most about 70 cells, at most about 60 cells, at most about 50 cells, at most about 40 cells, at most about 30 cells, at most about 20 cells, at most about 10 cells, at most about 9 cells, at most about 8 cells, at most about 7 cells, at most about 6 cells, at most about 5 cells, at most about 4 cells, at most about 3 cells, or at most about 2 cells. In one embodiment, the sample comprises only 1 cell.

It will be understood that the sample is typically a liquid sample. The sample may be an aqueous liquid sample, which may be buffered. Suitable buffers are known to the person skilled in the art. The buffer concentration in the sample is chosen such that a change in pH due to the presence of at least one circulating stromal cell is detectable upon incubation according to step (a). The buffer concentration in the sample may be at most about 50 mM, at most about 40 mM, at most about 30 mM, at most about 20 mM, at most about 10 mM, at most about 5 mM, at most about 4 mM, at most about 3 mM, at most about 2 mM, or at most about 1 mM.

In some embodiments, the pH of the sample may be from about 7.0 to 8.3, about 7.1 to 8.1, about 7.2 to 7.9, about 7.3 to 7.7, or about 7.4 to 7.6. In one embodiment, the pH of the sample is about 7.4.

In some embodiments, the concentration of the at least one molecule in the sample may be less than about 50 mM, less than about 40 mM, less than about 30 mM, less than about 20 mM, less than about 10 mM, less than about 9 mM, less than about 8 mM, less than about 7 mM, less than about 6 mM, less than about 5 mM, less than about 4 mM, less than about 3 mM, less than about 2 mM, less than about 1 mM, less than about 0.9 mM, less than about 0.8 mM, less than about 0.7 mM, less than about 0.6 mM, less than about 0.5 mM, less than about 0.4 mM, less than about 0.3 mM, less than about 0.2 mM, less than about 0.1 mM, or less than about 0.05 mM.

In one embodiment, the sample is in form of a droplet within a droplet-based microfluidic device. The droplet microfluidic device enables the manipulation of discrete fluidic packets in the form of picolitre or nanolitre droplets and addresses the need for lower costs, higher throughout and higher sensitivities at which the assays can be performed. The technique is well adapted to perform operations and manipulations in series, like encapsulation and screening. In particular, droplet microfluidic device allows to screen individual droplets using fluorescence-based techniques or mass spectrometry, to sort droplets from other droplets, to store them, to re-inject them into other microfluidic devices, to fuse droplets with other droplets and to culture cells in droplets. Thus, by encapsulating substantially all cells, for example, from about 1 mL to about 10 mL of blood into individual droplets, circulating stromal cells can be easily isolated thus providing inexpensive diagnostic applications and single cells for further studies.

Background references regarding droplet-based microfluidics, and how to prepare, manipulate (e.g. to sort droplets, control the temperature within droplets, fuse droplets, split droplets, mix the contents of droplets, and the like), and analyze droplets using microfluidics include A. B. Theberge et al., Angew. Chem. Int. Ed. 2010, volume 49, pages 5846-5868; and H.-D. Xi et al., Lab on a Chip 2019, volume 17, pages 751-771. For example, the fusion of droplets presents a way to contact chemicals with the sample being in the form of a droplet within a droplet-based microfluidic device. This is one way that allows for contacting the sample being in the form of a droplet with for example labels, staining solutions, and the like.

In one embodiment, the droplet is an aqueous droplet in a W/O emulsion. In one embodiment, the W/O emulsion has an oil component comprising a fluorous oil. In one embodiment, the oil component further comprises a surfactant. Suitable fluorous oil as those described in the state of the art include, for example, FC-77 and FC-40, which can be obtained from 3M™, and hydrofluoroethers (HFE), such as HFE 7500. Suitable surfactants are readily available and include, but are not limited to, RAN (available from Ran Biotechnologies), and Pico-Surf™ (available from Sphere Fluidics).

In other embodiments, the sample is present in a microwell or nanowell. In one embodiment, the microwell or nanowell is part of a microtiter or a nanotiter plate. Such microwells and nanowells, especially when they are part of a microtiter or nanotiter plate, can easily be analyzed in various ways, including nuclear magnetic resonance spectroscopy, fluorescence spectroscopy, UV/Vis spectroscopy, absorbance measurements, gas chromatography, mass spectrometry, and the like. In addition, samples present in microwells or nanowells can be easily and routinely manipulated, including heating, cooling, incubation, contacting with other chemicals, mixing, shaking, and the like.

In the method of the disclosure, the pH and/or concentration of at least one molecule selected from the group consisting of lactic acid, lactate ions, and pyruvate ions, within said sample, has been determined before step (c) of said method. It will be understood that the pH and/or concentration of the at least one molecule, within said sample, may have been determined by someone else than the person(s) carrying out the method of the disclosure, and/or at a different location than at which the method of the disclosure is carried out. The pH and/or concentration of the at least one molecule may be inferred by determining the pH and/or concentration of the at least one molecule of the fluid of which the sample is taken, provided that the pH and/or concentration of the at least one molecule are the same for the sample and the fluid. This is typically the case, since upon taking the sample from the fluid usually no further steps are carried out that influence the pH and/or concentration of the at least one molecule in the sample or the fluid.

In step (a) of the method the sample is incubated at a temperature from about 4° C. to about 42° C. for at least 1 minute to provide an incubated volume.

In some embodiments, the sample is incubated at a temperature from about 10° C. to about 41° C., from about 15° C. to about 40° C., from about 20° C. to about 39° C., from about 25° C. to about 38° C., or from about 30° C. to about 37° C. In one embodiment, the sample is incubated at a temperature of about 37° C.

In some embodiments, the sample is incubated for at least 2 minutes, at least 3 minutes, at least 4 minutes, at least 5 minutes, at least 6 minutes, at least 7 minutes, at least 8 minutes, at least 9 minutes, at least 10 minutes, at least 15 minutes, at least 20 minutes, at least 25 minutes, or at least 30 minutes.

In some embodiments, the sample is incubated for at most 1 day, at most 12 hours, at most 11 hours, at most 10 hours, at most 9 hours, at most 8 hours, at most 7 hours, at most 6 hours, at most 5 hours, at most 4 hours, at most 3 hours, at most 2 hours, at most 1 hour, or at most 45 minutes.

In step (b) of the method the pH and/or concentration of said at least one molecule, within the incubated volume, is determined. The pH and/or concentration of the at least one molecule can be determined by using any technique suitable for this purpose, known to the person skilled in the art. In one embodiment, in step (b) the pH and/or concentration of said at least one molecule is determined by using a pH-indicator and/or a fluorescent indicator. In one embodiment, the pH-indicator is a pH-sensitive dye or an indicator that changes its absorption/emission spectrum due to a change in pH. In one embodiment, the method of the disclosure further comprises a step of irradiating said incubated volume by a laser emitting at a visible wavelength, said change being a function of an emitted signal of said irradiated volume.

Suitable pH indicators are well-known to the person skilled in the art, and are readily available commercially. Examples of suitable pH indicators are SNARF-5F, pHrodo™ Green (Life Technologies), which fluoresces green at acidic pH, SNARFO-4F 5-(and-6)Carboxylic acid (Life Technologies), with the ratio between 580 nm and 640 nm fluorescence increasing at acidic pH, and pH-sensitive inorganic salt which aggregates to form microcrystals.

Suitable indicators for the concentration of the at least one molecule are also readily available. For example, lactic acid can be determined by fluorescent indicators (Fluoro lactate detection kit by Cell Technology, Inc.). Other ways for determining the concentration of the at least one molecule are also available. These include, but are not limited to, nuclear magnetic resonance (NMR) spectroscopy, liquid chromatography, gas chromatography, and quantitative mass spectrometry.

Furthermore, detection may be based on the mechanical properties of the incubated volume, or a change thereof as compared to the mechanical properties of the sample. In this case the indicator may for example comprise a monomer which undergoes pH-induced polymerization, or a polymer network of which the mechanical properties are pH dependent.

In step (c) of the method the pH and/or concentration of said at least one molecule determined in step (b) is compared to the pH and/or concentration of said at least one molecule, within said sample. Such a comparison can readily be made by a person skilled in the art. If such a comparison establishes a decrease in pH and/or an increase in concentration of said at least one molecule, then this indicates that at least one circulating stromal cell is present in said sample. It will be understood that herein, it is meant that the pH has decreased if the pH within the incubated volume is lower than within the sample prior to incubation. Similarly, it will be understood that the concentration of the at least one molecule has increased if said concentration is higher in the incubated volume than within the sample prior to incubation. Moreover, it will be understood that if no decrease in pH, and/or no increase in concentration of the at least one molecule is detected, this means that no circulating stromal cell was present in the sample.

In some embodiments, if the pH of the incubated volume is below about 7.3, below about 7.2, below about 7.1, below about 7.0, below about 6.9, below about 6.8, below about 6.7, below about 6.6, or below about 6.5, then this indicates that at least one circulating stromal cell is present in said sample.

In some embodiments, the concentration of the at least one molecule in the incubated volume is at least about 0.05 mM, at least about 0.1 mM, at least about 0.2 mM, at least about 0.3 mM, at least about 0.4 mM, at least about 0.5 mM, at least about 0.6 mM, at least about 0.7 mM, at least about 0.8 mM, at least about 0.9 mM, at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 20 mM, at least about 30 mM, at least about 40 mM, or at least about 50 mM.

In one embodiment, the fluid is an aqueous fluid. The fluid may further contain at least one solvent selected from the group consisting of ethanol, methanol, and dimethyl sulfoxide. In some embodiments, the fluid is a body fluid selected from the group consisting of blood, serum, lymph, pleural fluid, peritoneal fluid, cerebrospinal fluid, and urine. In one embodiment, the body fluid is blood. It will be understood that a body fluid can be taken from a subject in various ways available to the person skilled in the art, such as a medical professional.

In one embodiment, the fluid is obtained from a cell culture. Such a cell culture may contain or emulate a tumor microenvironment comprising stromal cell. It will be understood that stromal cells no longer associated with said tumor microenvironment, or emulated tumor microenvironment, are herein also referred to as circulating stromal cells. Such cell cultures may be used in in vitro studies of e.g. tumor development and/or metastasis. As used herein, cell cultures also include organoid cultures.

Optionally, the fluid has been processed, in whole or partially, before a sample is taken therefrom. For example, the fluid may have been subjected to one or more steps selected from the group consisting of dilution, concentration, heating, cooling, filtering, cell removal, and contacting with suitable chemical compounds. Suitable chemical compounds include, but are not limited to, buffers and salts (such as sodium chloride). Thus, while fluids as described herein include body fluids such as blood that are untreated prior to taking the sample, it also includes body fluids that have been processed as described herein.

In particular, the fluid may have been subjected to one or more cell removal steps before the sample is taken. Herein, “cell removal” includes cell depletion, and cell lysis. Typically, the cell removal steps are chosen such as not to remove a possibly present circulating stromal cell from the fluid. Cells to be removed are for example red blood cells, white blood cells, and tumor cells, in particular circulating tumor cells. In one embodiment, substantially all tumor cells have been removed from the fluid or the sample before step (a).

Various ways to selectively remove cells from a fluid are known to a person skilled in the art. For example, cell lysis can be performed by subjecting the fluid to an osmotic shock, for instance by contacting the fluid with a lysing buffer. In this way, cells such as red blood cells can be selectively lysed, and thus removed from the fluid. Cell depletion can for example be achieved by contacting the fluid with microbeads enriched with certain biomolecules, such as antibodies, that are specific towards certain cells, allow for these certain cells to bind to the microbeads, and then to remove the microbead-cell complex from the fluid. To remove white blood cells, CD45-enriched microbeads can be used. To remove red blood cells or red blood cell fragments (e.g. after red blood cell lysis) GlycophorinA-enriched beads can be used. The microbeads are for example magnetic, or are positively or negatively charged, so that they can easily be removed from the fluid in a way that is readily available to a person skilled in the art. These ways include, but are not limited to, passing the fluid over a suitable column, or through an open-ended tube of which the sides are for example encompassed by a magnet. Then, the microbeads bound to certain cells will for example remain on the column or within the open-ended tube, while the fluid passes, thus removing certain cells from the fluid. Alternatively, the fluid can be passed through a filter or over a size-exclusion column to separate cells by size. As is known to the person skilled in the art, different cell types have different sizes. Furthermore, cells can also be depleted from the fluid by cell sorting. To that end, cells can be selectively labelled, and sorted on the basis of the presence or absence of the label. Suitable labels include fluorophores, chromophores, enzymes, and radiolabels. Typically, these labels are attached to a biomolecule, such as an antibody, that specifically binds to a certain cell type. Cell sorting techniques are readily available to the skilled person.

In one embodiment, the method of the disclosure further comprises a step of contacting the fluid, the sample, or the incubated volume with at least one label selected from the group consisting of labels for circulating tumor cells, and labels for circulating stromal cells. The method of the disclosure may further comprise a step of detecting said at least one label. Contacting the fluid, the sample, or the incubated volume with said at least one label is advantageous when circulating tumor cells and circulating stromal cells need to be distinguished, for example when removing tumor cells from the fluid or sample by cell sorting.

Suitable labels for circulating tumor cells, and suitable labels for circulating stromal cells are readily available to the person skilled in the art. Suitable labels include fluorophores, chromophores, enzymes, and radiolabels. Typically, these labels are attached to a biomolecule, such as an antibody, that specifically binds to a target, which is present or absent, on either a tumor cell or a circulating stromal cell.

For example, targets for circulating tumor cells include cytokeratin 4, cytokeratin 5, cytokeratin 6, cytokeratin 8, cytokeratin 10, cytokeratin 13, cytokeratin 18, cytokeratin 19, and epithelial cell adhesion molecules (EpCAM). If these targets are present, that indicates that the cell is a circulating tumor cell. In addition, establishing a lack of CD45 expression (e.g. because a labelled antibody specific for CD45 does not bind to the cell) to further identify a cell as being a tumor cell.

Targets for circulating stromal cells typically vary per cell type, since no general marker is available. For example, targets that are present on circulating cancer-associated macrophage-like cells (CAMLs) include CD31, CD14, and CD68. Targets that are present on circulating cancer-associated vascular endothelial cells (CAVEs) include CD31, CD144, CD146, and vimentin. Targets that are present on circulating endothelial cells (CECs) include CD146, CD105, and vWF. Targets that are present on circulating cancer-associated fibroblasts include CD49b, CD87, and CD95. CD45 is a target that is typically absent on circulating stromal cells. Further information on targets for circulating tumor cells and/or circulating stromal cells is found in, inter alia, Adams and Cristofanilli, Liquid biopsies in Solid Tumors, Cancer Drug Discovery and Development 2017, Chapter 5: “Detecting and monitoring circulating stromal cells from solid tumors using blood-based biopsies in the twenty-first century: have circulating stromal cells come of Age?”; De Wit et al., Scientific Reports 2015, 5:12270; and Agorku et al., Front. Oncol. 2019, volume 9, article 716.

In one embodiment, the method of the disclosure further comprises the step of extracting the at least one cell from the incubated volume to provide at least one extracted cell. During or after extraction, cells from different samples may be pooled, wherein typically these different samples were obtained from the same fluid. For example, the extraction may be achieved by contacting the incubated volume in the form of a droplet in a W/O emulsion with an excess amount of aqueous liquid, for example after sorting the droplets. The unwanted droplets can be sorted and transported to a waste outlet, while the droplets of interest are led to a receptacle, such as a tube or a chamber, already containing said excess amount of aqueous liquid, or to which the excess amount of aqueous liquid can be added. For incubated volumes present in a microwell or nanowell, the at least one cell can be extracted by pipetting, and pooled with cells from other incubated volumes if necessary. Alternatively, the at least one extracted cell from a single incubated volume may be analyzed without pooling with other cells. Typically, the at least one extracted cell has been identified as a circulating stromal cell in step (c) of the method of the disclosure.

The extraction of the at least one cell from the incubated volume allows for subjecting the extracted cell or cells to further analysis, such as microscopic analysis to for example examine cell morphology, and various molecular biology analyses available to the person skilled in the art. In this way, different cell types can be further distinguished, if necessary, and/or the cells can be analyzed for research purposes, such as finding new biomarkers for cancer development.

In one embodiment, the method of the disclosure further comprises the step of subjecting the at least one extracted cell to an analysis selected from the group consisting of nucleotide analysis, epigenetic analysis, proteome analysis, and metabolome analysis. All these analyses can be routinely performed by a person skilled in the art.

In one embodiment, the nucleotide analysis genomic or transcriptomic analysis. Genomic analysis may be DNA sequencing, and transcriptomic analysis may be RNA sequencing, in particular mRNA sequencing. Genomic or transcriptomic analysis may serve to further identify the cell type of the at least one extracted cell. For genomic analysis, specific identifying genes can be directly analyzed. For transcriptomic analysis, the transcription products (such as mRNA) of such identifying genes can be analyzed. Such identifying genes are known to the person skilled in the art. By way of example, prostate cancer-related genes include AR, ERG, HOXB2, KLK3, PCA3, and TMPRSS2; circulating mesenchymal stem cell (MSC)-related genes include ANPEP, CD24, CD9, ITGB1, and TFRC; circulating endothelial cell-related genes include CD34, IGFBP7, MCAM, and VWF; circulating fibroblast-related genes include EGF, FGF2, FGFR1, FN1, PDGFB, PDGFC, and TGFB1; and circulating cancer-associated fibroblast genes include ESAM, GNG11, HIGD1B, COX4I2, CYGB, GJA4, and ENG.

Epigenetic analysis includes, but is not limited to, DNA methylation, RNA methylation, histone modifications, non-coding RNA analysis, and combinations thereof. Proteome analysis includes protein mass spectrometry, protein or peptide sequencing, protein crystallization and X- ray or cryo-TEM analysis, protein staining or labeling, and the like. Metabolome analysis includes liquid chromatography, gas chromatography, mass spectrometry, nuclear magnetic resonance spectroscopy, and combinations thereof.

### EXAMPLES

The method of the disclosure is further described in the following examples, which do not limit the scope of the methods of the disclosure described in the claims.

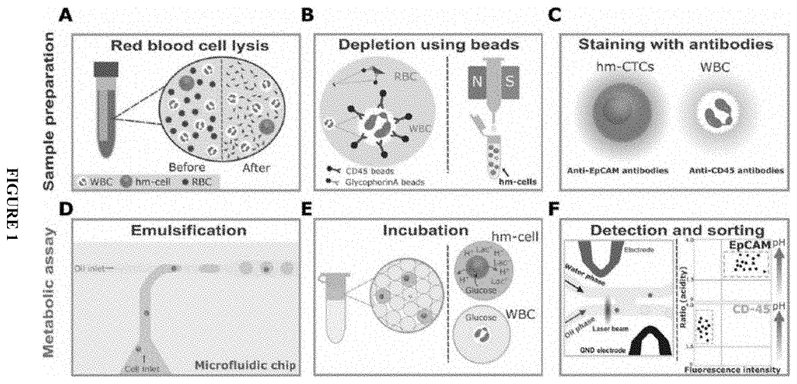

The study presented below describes one embodiment of the method of the disclosure. The workflow of this example is depicted in FIG. 1. In the study, blood was obtained from human subjects. The blood was processed, and samples of the processed blood were prepared by creating droplets in the form of W/O emulsions in a droplet-based microfluidic device. The samples contained a fluorescent pH indicator. Then, the sample was incubated to provide an incubated volume. The pH of the incubated volume was compared to the pH of the sample. Droplets that demonstrated a decrease in pH were sorted and further analyzed, showing that said droplets typically contained a circulating stromal cell. Thus, it was established that with a method according to the disclosure, based on a decrease in pH or increase in concentration of at least one molecule selected from the group of lactic acid, lactate ions, and pyruvate ions, within the sample upon incubation, circulating stromal cells can be detected.

### Study Design

A total of 82 participants including 56 patients with metastatic prostate cancer (all patients were castration resistant) and 26 healthy donors were recruited. Blood was typically withdrawn when the patient was progressing biochemically or radiographically but before the initiation of a new line of therapy. Two patients were not included in the Kaplan-Meier analysis due to lack of the date of the last follow-up.

### Clinical Samples

Blood collection for this study was approved by the medical ethics review committee under a biomarker protocol at the Radboud University Medical Center (CMO 2016-2793). Written informed consent was obtained from all patients according to good clinical practice for the use of their biomaterials as approved by the institutional review boards and local committees on research involving human subjects (ethics commission of the Radboud University Medical Center, Nijmegen, project number NL60249.091.16).

### Sample Preparation

Fresh whole blood (3 to 6 ml) was taken with a venous puncture and collected in K2-EDTA Vacutainer tubes (Becton Dickinson). A sample processing was started no longer than 30 min after a blood draw. Red blood cells (RBCs) were lysed by osmotic shock using BD Pharm Lyse Lysing Buffer (BD Biosciences, USA) following the manufacturer's protocol. Cells were then spun down, the supernatant was removed, and the pellet was resuspended in phosphate-buffered saline (PBS) (20012019, Thermo Fisher Scientific, USA) supplemented with 0.5% (w/v) bovine serum albumin (BSA) and 2 mM EDTA (final pH 7.4 adjusted by titration with NaOH solution). Tumor cells were subsequently enriched with CD45 and GlycophorinA depletion microbeads (Miltenyi Biotec, Germany) following the manufacturer's instructions. Briefly, 30 μl of CD45 microbeads and 10 μl of GlycophorinA microbeads were added to 150 μl of cell suspension and incubated for 15 min at 4° C. Then, 2 ml of PBS (with BSA and EDTA) was used to wash the walls of falcon, and the sample was spun down to remove the supernatant. The pellet was gently resuspended in 500 μl of PBS (supplemented with 0.5% BSA and 2 mM EDTA) and loaded through LD column (Miltenyi Biotec, Germany), according to the protocol attached by the manufacturer. In the case of staining, the enriched sample was incubated with Brilliant Stain Buffer (the buffer mixed with the sample in the ratio 1:2; 563794, BD Horizon, USA), EpCAM BV421 mouse anti-human (1:100 dilution; 563180, BD Horizon), and CD45 BV480 mouse anti-human (1:100 dilution; 566156, BD Horizon). Last, the sample was resuspended in solution composed of Joklik's modified minimum essential medium (MEM) (M0518, Sigma- Aldrich, USA) supplemented with 0.1% BSA (w/v), 2 mM EDTA (final pH 7.4 adjusted by addition of NaOH solution) mixed with 15% (v/v) OptiPrep Density Gradient (Sigma-Aldrich, USA), and 11 μM SNARF-5F (Thermo Fisher Scientific, USA) before emulsification. All solutions were filtered through sterile polyvinylidene difluoride filters (pore size, 0.22 μm) before use. PBS and Eagle's MEM (EMEM)-based medium were stored on ice before cell resuspension and washing steps.

### Microfluidic Device Fabrication for Emulsification and Sorting

Microfluidic devices were produced by using photo and soft lithography. Silicon wafers were spin-coated with a uniform layer of SU8-2025 photoresist (MicroChem Co., USA), soft-baked, ultraviolet-exposed through transparency mask (JD Photo-Tools, UK), baked again post-exposure, developed, and hard-baked according to the manufacturer's protocol (MicroChem Co., USA). After production, the height of the structures was measured by Dektak profilometer. The emulsification devices had the height of 25 μm, and the sorting devices had the height of 30 μm. These wafers were used as masters for the polydimethylsiloxane (PDMS) devices. PDMS prepolymer and cross-linking agent were mixed at a 10:1 ratio (w/w) and poured on the master bearing the microchannel structure. The PDMS was further degassed (for 30 min) and cured at 65° C. for at least 2 hours. Then, the PDMS replica was cut out from the master, and the inlets and outlets were punched using a biopsy puncher of 1 mm inner diameter (pfm medical, USA). Thereafter, the replica and a glass slide were carefully washed first with a mixture of soap and water and then with ethanol. The clean replica was bonded to the glass slide after oxygen plasma treatment (Femto, Diener electronic). The channels of emulsification device were rendered hydrophobic by silanization with 5% 1H,1H,2H,2H-perfluorooctlytriethoxysilane (Sigma-Aldrich, USA) in FC-40 (Sigma-Aldrich, USA). In the case of sorting devices, two phases were run parallelly to modify channels: (i) 5% solution of 1H,1H,2H,2H-perfluorooctyltriethoxysilane (Sigma-Aldrich, USA) in FC-40 (v/v) to modify oil phase channel and (ii) 1% (v/v) 11-bromoun decyldimethylchlorosilane (Fluorochem, UK) in cyclohexane to modify water phase channel. During modification, these two phases were run in a co-flow manner. Then, the chip was flushed with HFE-7500 oil 3M (Fluorochem, UK). The emulsification and sorting devices were incubated at 95° C. overnight. Electrodes in a sorting chip were prepared immediately before analysis. Small rods (2 to 3 mm) of indium (51%), bismuth (32.5%), and tin (16.5%) alloy (Indium Corporation of America, IPN 51962) were introduced into punched holes, and a device was placed on the hot plate. Once alloy started to melt, tinned copper wires (Rowan Cable Products Ltd., TCW21 1230994) were placed in the channels to provide the contact. The electrodes in the sorting chip were observed under the microscope whether they completely filled channels.

### Droplet Production and Incubation

A sample, cells resuspended in Joklik's modified EMEM with BSA, EDTA, OptiPrep, and SNARF-5F, was emulsified immediately after preparation. Monodisperse water-in-oil droplets were generated by using the emulsification device described above with 20-μm-wide T-junction. The composition of the continuous phase was 2.5% (w/w) of RAN (RAN Biotechnologies, USA) in HFE-7500. The flow rate for the continuous phase was 700 μl/hour, and the flow rate for the dispersed phase was 300 μl/hour, which allowed the production of 30-pl droplets. Emulsification step was performed at 4° C. to decrease cell metabolism and prevent the secretion of lactate acid in the bulk. The collected emulsion was incubated in 37° C. in the thermal block for 30 min. After the incubation step, the sample was immediately placed on ice.

### Detection and Sorting

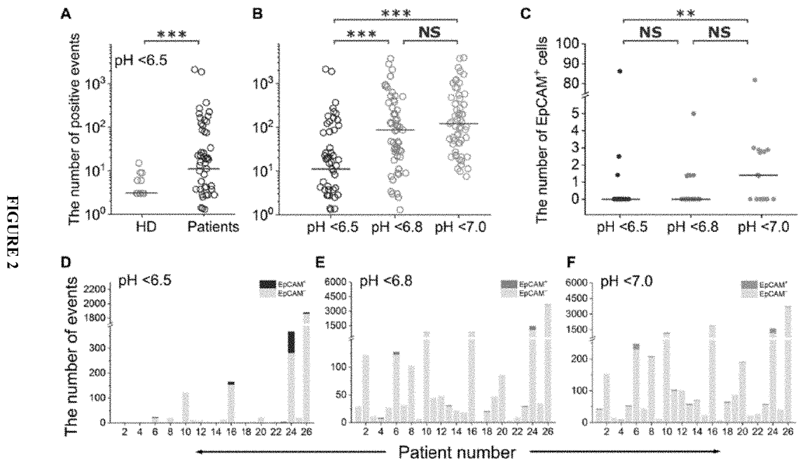

A sorting chip with three inlets (water phase, droplet, and oil spacer) and three electrodes embedded in PDMS was used for droplet reinjection and sorting process. The water phase was composed of 50 mM tris (15567027, Thermo Fisher Scientific, USA) and 150 mM NaCl (S3014, Sigma-Aldrich, USA) in nuclease-free water (Ambion, Thermo Fisher Scientific, USA). The oil phase consisted of 0.5% (w/w) of RAN in HFE-7500 oil. The emulsion was loaded to the tubing with a previously made air spacer to avoid spreading droplets in an HFE-7500-loaded syringe. The tubing with emulsion was wrapped around the ice pack to prevent the increase of temperature and further secretion of lactic acid by the cells. The flow rates of the water phase, oil spacer, and emulsion were ˜3000, 300, and 30 μl/hour, respectively. Solutions were pumped using neMESYS (CETO-NI GmbH, Germany) syringe pumps. First, the oil and the water phase were run through the chip. As soon as the flow was stabilized and the water phase was not dripping into the oil channel, droplets were introduced into the channel, flowing one by one. A modified microscope setup for detection of acidic droplets is described elsewhere (F. D. Ben et al., Angew. Chem. Int. Ed. Engl., 2016, volume 55, pages 8581-8584). An inverted microscope (Olympus IX71, Japan) was used to detect droplets with acidified pH. Expanded (2×) laser beam (408 Argon-ion Cyonics) was focused down with a cylindrical lens across the microfluidic channel. The fluorescence signal was collected with a 40× objective (Olympus LUCPlanFLN, 40×/0.60), and four fluorescence bands (450, 525, 580, and 630 nm) were acquired using a cRIO-9024 acquisition system (National Instruments) at a scan rate of 105 Hz. In-house written LabVIEW software was used to control the acquisition system to detect all data points of droplets over a given threshold and provides trigger pulse for image capture.
The voltage generation setup consisted of a 33220A arbitrary waveform generator (Agilent, USA) and high-voltage power amplifier (TREK model 2220, TREK Inc., USA). The function generator was used to generate sine voltage waveform of the electric field, typically at 3.5 V (Vpp), 5 kHz (output five cycles) that was amplified by a factor of 200. The count of detected hm-cells was normalized to 7.5 ml of blood for convenient comparison with the CellSearch method. The results of this analysis are shown in FIG. 2.

### Statistical Analysis

Pearson's correlation (r) was tested for associations between hm-cells and other continuous baseline prognostic variables using SPSS software platform, and the t test was used for differences between hm-cells and dichotomous variables.

### Survival Probability Plot

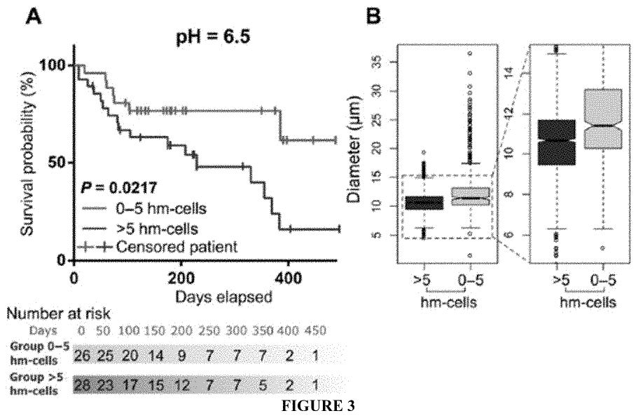

Median overall survival (OS) was estimated using the Kaplan-Meier method, and multivariable Cox proportional hazard models were tested for associations with OS. The Kaplan-Meier survival plot was obtained using GraphPad Prism 8 (version 8.0.1). Data were censored at the date of the last follow-up when death had not occurred. Log-rank (Mantel-Cox) test was used to compare the survival curves. The optimal cutoff for prognostication purposes was evaluated based on c-statistics. P<0.05 was considered significant. The Kaplan-Meier plot shown in FIG. 3A is based on data for 54 of 56 patients due to missing clinical information. Further analysis of the results is depicted in FIG. 3B.
Library Preparation for Single-Cell mRNA Sequencing
The cells after sorting were collected in four to five sterile 1.5-ml low-binding Eppendorf tubes and kept at 4° C. The cell suspension obtained after sorting was concentrated by centrifugation (5 min, 500 relative centrifugal force, 4° C.), pooled together, and subsequently centrifuged, yielding in 100 μl. Single cells were distributed in 96-well plates with barcoding primers, by limited dilution, by pipetting 1 μl of cell suspension solution in each well. To avoid doublets in wells, we used λ=0.5 to 1 cell per well. If more than 96 cells were sorted out, then an additional plate was used for barcoding of the mRNA material. The plates with the cells and barcoding primers were stored at −80° C. until further processing. The library preparation was based on CEL-Seq2 (T. Hashimshony et al., Genome Biol. 2016, volume 17, page 77). Prepared libraries were sequenced with NextSeq500 (targeting 10 million reads per sample).
Single-Cell mRNA Data Processing
From the FASTQ files generated by the Illumina sequencer, transcriptome count tables were generated using the published CEL-Seq2 pipeline (T. Hashimshony et al., Genome Biol. 2016, volume 17, page 77) with some minor modifications. To allow compatibility with the pipeline, read1 and read2 were swapped. Briefly, first, the reads were demultiplexed to be assigned each to a specific cell. Second, they were aligned to the known human genome using Bowtie. Last, UMIs (Unique Molecular Identifiers) for each cell were counted and a count table was generated. Further processing of the data was performed in R software using Seurat V3 package (A. Butler et al., Nat. Biotechnol. 2018, volume 36, pages 411-420). All cells from the different plates analyzed were merged in one Seurat object. Quality check excluded cells with less than 200 genes per cell and with more than 5000 genes per cell detected from sequencing. The count of transcripts per cells was normalized using Seurat in the form of reads per million.

### Differential Expression and Pathways Analysis

Single cells were devised in two clusters in an unsupervised manner using t-SNE in Seurat package. The single-cell counts were normalized and scaled to remove unwanted sources of variation. Analysis was run on 13 significant principal components, and clusters were generated from Seurat package setting the resolution variable to 0.5. Last, the DEGs between the clusters were identified in an unbiased way using Wilcoxon rank sum test. Only genes detected in a minimum fraction of 30% of cells in one of the two populations and that showed, on average, at least 0.25-fold difference (log scale) between the two groups of cells were tested. Gene set overrepresentation analysis was performed using UniProt database and KEGG. t-SNE plots with the collective expression of known genes. The cumulative expression of genes with known functions or characteristics of a cell type was plotted over the t-SNE. For the MSCs, the genes presented as positive cell markers on LifeMap Discovery were chosen. For the fibroblasts, genes related to ECM components (FN1 or fibronectinl), growth factors, and cytokines (EGF, FGF2, PDGFB, PDGFC, and TGFB1) and related to receptors and other membrane-bound proteins (FGFR1) were used, which were expressed in our dataset and were mostly used to identify CAFs in literature (V. S. LeBleu et al., Dis. Model. Mech. 2018, volume 11, dmm029447). Last, for the endothelial genes, we summed up the most frequently used markers in the various CEC assays (J. Kraan et al., Drug Discov. Today 2012, volume 17, pges 710-717), when expressed in our dataset.

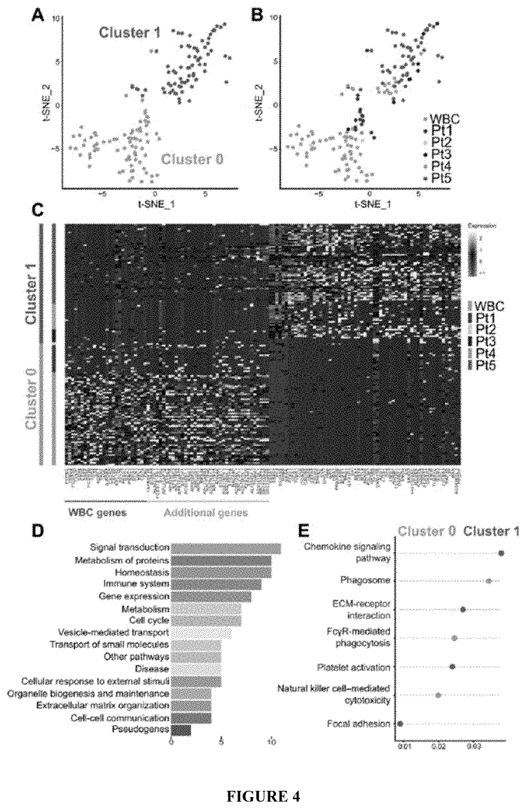

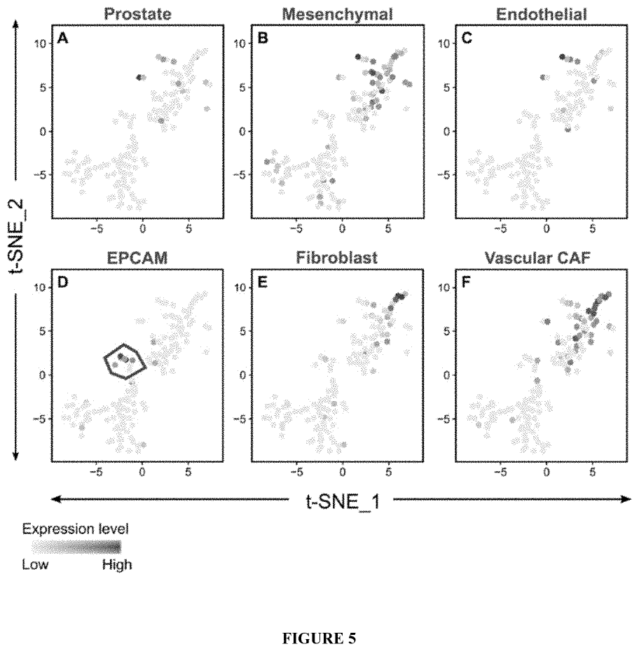

The results of these analyses are shown in FIGS. 4 and 5.

These analyses showed that circulating stromal cells are able to acidify their environment. Thus, this example shows that comparing the pH and/or concentration of the at least one molecule determined for the incubated volume to the pH and/or concentration of said at least one molecule, within said sample; allows for a circulating stromal cell to be identified.

### OTHER EMBODIMENTS

It is to be understood that while the methods of the disclosure have been described in conjunction with the detailed description thereof, the foregoing description is intended to illustrate and not limit the scope of the methods of the disclosure, which is defined by the scope of the appended claims. Other aspects, advantages, and modifications are within the scope of the following claims.

### REFERENCES

Matula, Kinga, et al. “Microfluidic isolation of metabolically active circulating tumor cells and circulating stromal cells.”μTAS 2019. The 23^{rd} International Conference on Miniaturized Systems for Chemistry and Life Sciences. 29 Oct. 2019. 27 pages.

Rivello, Francesca, et al. “Probing single-cell metabolism reveals prognostic value of highly metabolically active circulating stromal cells in prostate cancer.” Science Advances 6.40 (2020): eaaz3849.

Rivello, Francesca, et al. “Supplementary Materials for Probing single-cell metabolism reveals prognostic value of highly metabolically active circulating stromal cells in prostate cancer.” Science Advances 6.40 (2020) advances.sciencemag.org/cgi/content/full/6/40/eaaz3849/DC1. 48 pages.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

1. A method for detecting at least one circulating stromal cell in a sample of about 10 pL to about 10 nL of a fluid, wherein said sample comprises at least one cell, and wherein the pH and/or concentration of at least one molecule selected from the group consisting of lactic acid, lactate ions, and pyruvate ions, within said sample, has been determined before step (c) of said method;
wherein said method comprises the steps of:
(a) incubating said sample at a temperature from about 4° C. to about 42° C. for at least 1 minute to provide an incubated volume;
(b) determining the pH and/or concentration of said at least one molecule, within said incubated volume; and
(c) comparing the pH and/or concentration of said at least one molecule determined in step (b) to the pH and/or concentration of said at least one molecule, within said sample;
wherein a decrease in pH and/or an increase in concentration of said at least one molecule, indicates said at least one circulating stromal cell is present in said sample.

2. The method according to claim 1, wherein the fluid is an aqueous fluid.

3. The method according to claim 1, wherein the fluid is a body fluid selected from the group consisting of blood, serum, lymph, pleural fluid, peritoneal fluid, cerebrospinal fluid, and urine; and wherein optionally the body fluid has been diluted.

4. The method according to claim 1, wherein the fluid is obtained from a cell culture.

5. The method according to claim 1, wherein said sample is in form of a droplet within a droplet-based microfluidic device.

6. The method according to claim 5, wherein said droplet is an aqueous droplet in a W/O emulsion.

7. The method according to claim 6, wherein said W/O emulsion has an oil component comprising a fluorous oil.

8. The method according to claim 7, wherein said oil component further comprises a surfactant.

9. The method according to claim 1, wherein said sample is present in a microwell or nanowell.

10. The method according to claim 9, wherein said microwell or nanowell is part of a microtiter or a nanotiter plate.

11. The method according to claim 1, wherein in step (b) the pH and/or concentration of said at least one molecule is determined by using a pH-indicator and/or a fluorescent indicator.

12. The method according to claim 11, wherein said pH-indicator is a pH-sensitive dye or an indicator that changes its absorption/emission spectrum due to a change in pH.

13. The method according to claim 12, further comprising a step of irradiating said incubated volume by a laser emitting at a visible wavelength, said change being a function of an emitted signal of said irradiated volume.

14. The method according to claim 1, further comprising a step of contacting the fluid, the sample, or the incubated volume with at least one label selected from the group consisting of labels for circulating tumor cells, and labels for circulating stromal cells.

15. The method according to claim 14, further comprising a step of detecting said at least one label.

16. The method according to claim 1, further comprising the steps of
extracting the at least one cell from the incubated volume to provide at least one extracted cell; and
subjecting the at least one extracted cell to an analysis selected from the group consisting of nucleotide analysis, epigenetic analysis, proteome analysis, and metabolome analysis.

17. The method according to claim 16, wherein the nucleotide analysis is selected from the group consisting of mRNA sequencing, and DNA sequencing.

18. The method according to claim 16, wherein the epigenetic analysis is selected from the group consisting of mRNA methylation analysis, DNA methylation analysis, histone modification analysis, and non-coding RNA analysis.

19. The method according to claim 1, wherein substantially all tumor cells have been removed from the fluid or the sample before step (a).

20. The method according to claim 1, wherein the at least one circulating stromal cell is selected from the group consisting of circulating endothelial cells, circulating mesenchymal stem cells, and circulating cancer-associated fibroblasts.
